(19) United States
(12) Patent Application Publication
(10) Pub. No.: US 2024/0199455 A1
(43) Pub. Date: Jun. 20, 2024

(54) REMOVAL OF TOXIC OXYANIONS BY CO-CRYSTALLIZATION WITH SULFATE

(71) Applicant: UT-Battelle, LLC, Oak Ridge, TN (US)

(72) Inventors: Radu Custelcean, Knoxville, TN (US); Jeffrey Einkauf, Knoxville, TN (US)

(21) Appl. No.: 18/371,528

(22) Filed: Sep. 22, 2023

Related U.S. Application Data
(60) Provisional application No. 63/428,783, filed on Nov. 30, 2022.

(51) Int. Cl. C02F 1/52; C07C 279/00; C07D 213/53

(52) U.S. Cl. CPC C02F 1/5272; C07C 279/00; C07D 213/53; C02F 2101/106

(57) ABSTRACT

Method for removing toxic oxyanions (e.g., selenate) from an aqueous source as follows: (i) dissolving an oxyanion precipitating compound (OPC) in the aqueous source to result in precipitation of a salt containing sulfate, toxic oxyanion, and the OPC, wherein the sulfate in the aqueous source is in a molar concentration at least equal to the total molar concentration of the toxic oxyanion, and the OPC is included in the aqueous source in a molar concentration equal to or greater than the total molar concentration of sulfate and toxic oxyanion; and (ii) removing the precipitated salt from the aqueous source to result in a supernatant containing a substantially lower concentration of the toxic oxyanion compared to the aqueous source.

### CROSS REFERENCE TO RELATED APPLICATION

The present application claims benefit of U.S. Provisional Application No. 63/428,783, filed on Nov. 30, 2022, all of the contents of which are incorporated herein by reference.

### STATEMENT REGARDING FEDERALLY SPONSORED RESEARCH

This invention was made with government support under Prime Contract No. DE-AC05-00OR22725 awarded by the U.S. Department of Energy. The government has certain rights in the invention.

### FIELD OF THE INVENTION

The present invention generally relates to methods for removing undesirable species from aqueous solutions, such as removal of toxic oxyanion species. The present invention is more specifically directed to methods for in which an oxyanion precipitating compound, or more particularly, a bis(iminoguanidinium) compound, is used to precipitate an oxyanion species from an aqueous source.

### BACKGROUND

Selenium is an essential element for most living organisms at trace levels (40-400 μg/day for humans), but becomes toxic at higher concentrations. Selenium oxyanions, selenate (SeO₄²⁻) and selenite (SeO₃²⁻), are introduced into the environment from both natural and anthropogenic sources, such as mining and fossil fuel combustion, and have high solubility and mobility in aquatic environments. Approximately 40% of the United States population receives groundwater-sourced drinking water through either public supply or private wells, providing a source for potential exposure to these toxic oxyanions. As such, the United States Environmental Protection Agency (EPA) has set limits on the acceptable concentration of Se in drinking water to 0.05 mg/L (N. Kozai et al., J. Mater. Res., 17(12), 2993-2996, 2002). Se is even more toxic to aquatic ecosystems, which has led to stringent regulatory discharge limits of 5 μg/L (5 ppb) set by the EPA for Se-containing wastewaters. However, removal of Se oxyanions from wastewaters comes with major technical challenges associated with their trace-level ppb concentrations, competing ions present in much higher concentrations, slow kinetics, and high variability in environmental conditions such as ionic strength, pH, and temperature.

Physical treatment is based primarily on membrane filtration. This method has high capital and operational cost, high maintenance, and typically requires pretreatment of the wastewater to remove suspended solids and minimize scaling and fouling. Ion exchange is commonly employed in industrial wastewater treatment, but it has found limited success for Se removal due to competition with other anions that are present in much higher concentrations. Ferrihydrite adsorption is another chemical method available for Se removal that involves the precipitation of ferrihydrite from ferric salts followed by adsorption of Se oxyanions on the ferrihydrite solid particles. However, this method does not strongly bind selenate. Coprecipitation methods involve the inclusion of Se oxyanions in crystals or insoluble salts such as barite. However, this method is highly dependent on the concentration of competing anions, ionic strength, and solution pH. Inorganic and microbial approaches utilize reductive methods to produce elemental selenium or iron selenides, but more work is needed to elucidate the factors that control the formation and selectivity of various reduced Se phases (e.g., ferroselite, dzharkenite, Se-substituted pyrite). Adsorption methods are more effective for selenite separation than for selenate, and may leach the oxyanion back into solution upon changes in the surrounding environment. Thus, other more effective and lower cost methods are needed for removing selenium and other toxic oxyanions from water sources.

### SUMMARY

The present disclosure is foremost directed to a co-crystallization method for removing toxic oxyanions (e.g., selenate, selenite, molybdate, arsenate, and/or chromate) from an aqueous source containing sulfate and at least one toxic oxyanion. The aqueous source may be, for example, drinking water, groundwater, industrial effluent, or wastewater. The method is advantageously capable of quantitative removal of toxic oxyanions by co-crystallization of sulfate and the toxic oxyanion. In the method, a specialized bis-iminoguanidinium oxyanion precipitating compound is dissolved in the aqueous source to result in precipitation of a substantially insoluble salt containing sulfate, at least one toxic oxyanion, and the oxyanion precipitating compound. For purposes of the invention, the sulfate in the aqueous source is in a molar concentration equal to or greater than the total molar concentration of the toxic oxyanion, and the oxyanion precipitating compound is included in the aqueous source in a molar concentration equal to or greater than the total molar concentration of sulfate and toxic oxyanion. The co-crystallization method described above can be used as an effective remediation strategy for removing toxic oxyanions, particularly selenate and/or selenite oxyanions, leading to their substantial or nearly quantitative removal from wastewaters, down to ppb and sub-ppb levels. In some embodiments, the toxic oxyanion is or includes selenate, selenite, or combination thereof. In typical embodiments, the sulfate is present in the aqueous source in a concentration at least or more than 100, 500, or 1000 times the total concentration of toxic oxyanion. In some embodiments, the oxyanion precipitating compound is included in the aqueous source in at least twice, three times, or four times the molar concentration of the total molar concentration of sulfate and toxic oxyanion

By removal of the salt containing co-crystallized sulfate and toxic oxyanion, the toxic oxyanion from the water can be easily removed. The process described herein is advantageously straight-forward and cost-efficient while at the same time removing a substantial amount or substantially all of the toxic oxyanion from an aqueous source without requiring pressure, nanofiltration, pre-concentration, and organic solvents. The process described herein operates by simple self-assembly of the precipitating compounds and co-crystallization of sulfate and the toxic oxyanion, thereby circumventing the need for elaborate syntheses of compounds that precipitate the oxyanion directly without the aid of self-assembly. Moreover, the bis-iminoguanidinium oxyanion precipitating compounds described herein can be advantageously recycled and re-used in the oxyanion removal process.

The oxyanion precipitating compound has the following structure:

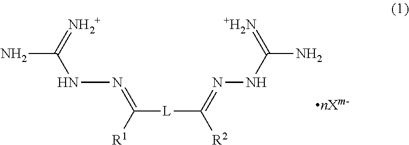

In the above Formula (1), L is a bond or a hydrocarbon linker containing 1-12 carbon atoms (e.g., a ring, such as an aromatic or heteroaromatic ring) and optionally containing one or more heteroatoms selected from O, N, and S; R¹ and R² are independently selected from H and hydrocarbon groups containing 1-12 carbon atoms (e.g., methyl, ethyl, n-propyl, or isopropyl); X^{m-} is an anionic species with a magnitude of charge m, where m is an integer of at least 1, provided that X^{m-} is an anionic species exchangeable with sulfate and the toxic oxyanion in the aqueous source before the oxyanion precipitating compound contacts the aqueous source in step (i), and X^{m-} represents a co-crystallized combination of sulfate and the toxic oxyanion after the oxyanion precipitating compound contacts the aqueous source and forms the resulting oxyanion salt in step (i) and is removed from the aqueous source in step (ii) to result in a supernatant containing at least 90% lower concentration of the toxic oxyanion compared to the aqueous source. The subscript n is an integer of at least 1, provided that n×m=2. In some embodiments, X^{m-} is a halide species selected from Cl⁻, Br⁻, and I⁻ before the oxyanion precipitating compound is dissolved in the aqueous source in step (i).

In some embodiments of Formula (1), the oxyanion precipitating compound has the following structure:

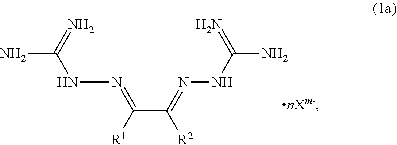

wherein R¹, R², n, and X^{m-} are as defined above under Formula (1).

In other embodiments of Formula (1), the oxyanion precipitating compound has the following structure:

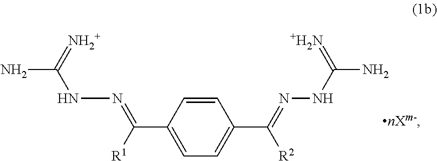

wherein R¹, R², n, and X^{m-} are as defined above under Formula (1).

In other embodiments of Formula (1), the oxyanion precipitating compound has the following structure:

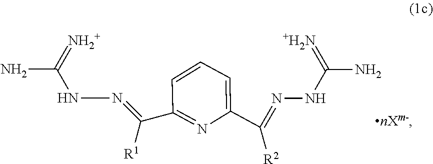

wherein R¹, R², n, and X^{m-} are as defined above under Formula (1).

In any of the above embodiments, the method may further include a recycling step of the oxyanion precipitating compound as follows: (iii) treating the salt obtained in step (ii) with a base to form an uncharged precursor of the oxyanion precipitating compound along with simultaneous release and isolation of sulfate and toxic oxyanion, followed by acidification of the uncharged precursor of the oxyanion precipitating compound with an acid of the formula HX, wherein X is an anionic species exchangeable with sulfate and the toxic oxyanion in said aqueous source to re-form the oxyanion precipitating compound of Formula (1). In some embodiments, X is Cl, Br, or I.

In any of the above embodiments, the method may further include adding sulfate to the supernatant in step (ii) to precipitate additional salt containing the oxyanion precipitating compound; combining the additional salt with the salt already removed in step (ii) to form a total amount of precipitated salt; and subjecting the total amount of oxyanion precipitated salt to a recycling process, as follows: (iii) treating the total amount of precipitated salt with a base to form an uncharged precursor of the oxyanion precipitating compound along with simultaneous release and isolation of sulfate and toxic oxyanion, followed by acidification of the uncharged precursor of the oxyanion precipitating compound with an acid of the formula HX, wherein X is an anionic species exchangeable with sulfate and the toxic oxyanion in said aqueous source to re-form the oxyanion precipitating compound of Formula (1).

In some embodiments, the method further includes adding sulfate to the aqueous source. In a first set of embodiments, the aqueous source initially contains a concentration of sulfate that is less than 100 times the total concentration of toxic oxyanion, and sulfate is added to the aqueous source to result in the concentration of sulfate being at least 100 times the total concentration of toxic oxyanion. In a second set of embodiments, the aqueous source initially contains a concentration of sulfate that is less than 500 times the total concentration of toxic oxyanion, and sulfate is added to the aqueous source to result in the concentration of sulfate being at least 500 times the total concentration of toxic oxyanion. In a third set of embodiments, the aqueous source initially contains a concentration of sulfate that is less than 1000 times the total concentration of toxic oxyanion, and sulfate is added to the aqueous source to result in the concentration of sulfate being at least 1000 times the total concentration of toxic oxyanion.

In any of the above embodiments, the aqueous source may contain a concentration of toxic oxyanion of at least or up to 500 ppb and the supernatant in step (ii) contains a toxic oxyanion concentration of no more than or less than 50, 20, 10, or 5 ppb. In other embodiments, the aqueous source may contain a concentration of toxic oxyanion of at least or up to 200 ppb and the supernatant in step (ii) contains a toxic oxyanion concentration of no more than or less than 20, 10, or 5 ppb. In other embodiments, the aqueous source may contain a concentration of toxic oxyanion of at least or up to 100 ppb and the supernatant in step (ii) contains a toxic oxyanion concentration of no more than or less than 10, 5, 2, or 1 ppb. In other embodiments, the aqueous source may contain a concentration of toxic oxyanion of at least or up to 50 ppb and the supernatant in step (ii) contains a toxic oxyanion concentration of no more than or less than 5, 2, or 1 ppb.

### BRIEF DESCRIPTION OF THE DRAWINGS

FIG. 1. Structures of bis(iminoguanidinium) (BIG) cations, used as chloride salts, for crystallization studies with sulfate, selenate, and selenite oxyanions: glyoxal-bis(iminoguanidinium) (GBIG); methylglyoxal-bis(iminoguanidinium) (MGBIG); diacetal-bis(iminoguanidinium) (DABIG); benzenedialdehyde-bis(iminoguanidinium) (BBIG); and pyridinedialdehydebis(iminoguanidinium) (PyBIG).

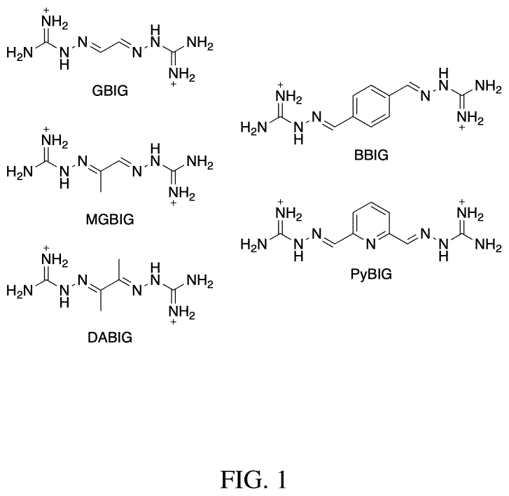

FIGS. 2a-2d. FIGS. 2a and 2b are graphs showing removal efficiencies of 5 mM sulfate, selenate, or selenite with increasing concentrations of BBIG (FIG. 2a) or PyBIG (FIG. 2b). FIGS. 2c and 2d are graphs showing removal efficiency of solutions containing both sulfate and selenate with increasing concentrations of BBIG (FIG. 2c) or PyBIG (FIG. 2d).

FIGS. 3a-3d. Graphs showing oxyanion removal with 1 equiv of BBIG (FIG. 3a) or PyBIG (FIGS. 3b) and 2 equiv of BBIG (FIG. 3c) or PyBIG (FIG. 3d) from aqueous solutions containing 2.5 mM sulfate and decreasing concentrations of selenate.

FIGS. 4a-4d. Graphs showing removal efficiency of sulfur and selenium from simulated groundwater with BBIG (FIG. 4a) and PyBIG (FIG. 4b), and from simulated leachate with BBIG (FIG. 4c) and PyBIG (FIG. 4d). The US EPA regulatory discharge limit target of 5 ppb is depicted as a dashed line.

FIG. 5. Schematic showing separation cycle for sulfate/selenate removal by co-crystallization with a guanidine ligand. (B) Co-crystallization of BBIG·SO₄/SeO₄. (C) Filtration of BBIG·SO₄/SeO₄ crystals. (D) Guanidine ligand recovery by neutralization with NaOH and crystallization of neutral BBIG; sulfate and selenate are recovered as concentrated aqueous solutions. (E) Regeneration of the guanidine chloride salt, which can be recycled.

### DETAILED DESCRIPTION

As used herein, the term “hydrocarbon group” (also denoted by the group R) is defined as a chemical group containing at least carbon and hydrogen atoms. In some embodiments, R is composed of solely carbon and hydrogen. In other embodiments, R is composed of carbon and hydrogen with optional substitution of the hydrocarbon group with one or more fluorine atoms to result in partial or complete fluorination of the hydrocarbon group. In other embodiments, R is composed of carbon and hydrogen with optional substitution with fluorine along with the presence of one or more heteroatoms selected from oxygen, nitrogen, and sulfur atoms. In different embodiments, one or more of the hydrocarbon groups contain, for example, 1, 2, 3, 4, 5, 6, 7, 8, 9, 10, 11, or 12 carbon atoms, or a number of carbon atoms within a particular range bounded by any two of the foregoing carbon numbers (e.g., 1-12, 1-8, 1-6, 1-5, 1-4, 1-3, 2-12, 2-8, 2-6, 2-5, 2-4, or 2-3 carbon atoms). Hydrocarbon groups in different compounds described herein, or in different generic groups of a compound, may possess the same or different number (or preferred range thereof) of carbon atoms. For example, any one of R¹, R², R³, and/or R⁴ in any of the generic formulas disclosed herein may independently contain a number of carbon atoms within any of the ranges provided above.

In a first set of embodiments, the hydrocarbon group (R) is a saturated and straight-chained group, i.e., a straight-chained (linear) alkyl group. Some examples of straight-chained alkyl groups include methyl, ethyl, n-propyl, n-butyl, n-pentyl, n-hexyl, n-heptyl, n-octyl, n-nonyl, n-decyl, n-undecyl, and n-dodecyl groups.

In a second set of embodiments, the hydrocarbon group (R) is saturated and branched, i.e., a branched alkyl group. Some examples of branched alkyl groups include isopropyl (2-propyl), isobutyl (2-methylprop-1-yl), sec-butyl (2-butyl), t-butyl (1,1-dimethylethyl-1-yl), 2-pentyl, 3-pentyl, 2-methylbut-1-yl, isopentyl (3-methylbut-1-yl), 1,2-dimethylprop-1-yl, 1,1-dimethylprop-1-yl, neopentyl (2,2-dimethylprop-1-yl), 2-hexyl, 3-hexyl, 2-methylpent-1-yl, 3-methylpent-1-yl, isohexyl (4-methylpent-1-yl), 1,1-dimethylbut-1-yl, 1,2-dimethylbut-1-yl, 2,2-dimethylbut-1-yl, 2,3-dimethylbut-1-yl, 3,3-dimethylbut-1-yl, 1,1,2-trimethylprop-1-yl, 1,2,2-trimethylprop-1-yl groups, isoheptyl, isooctyl, and the numerous other branched alkyl groups having up to 12 carbon atoms, wherein the “1-yl” suffix represents the point of attachment of the group.

In a third set of embodiments, the hydrocarbon group (R) is saturated and cyclic, i.e., a cycloalkyl group. Some examples of cycloalkyl groups include cyclopropyl, cyclobutyl, cyclopentyl, cyclohexyl, cycloheptyl, and cyclooctyl groups. The cycloalkyl group can also be a polycyclic (e.g., bicyclic) group by either possessing a bond between two ring groups (e.g., dicyclohexyl) or a shared (i.e., fused) side (e.g., decalin and norbornane).

In a fourth set of embodiments, the hydrocarbon group (R) is unsaturated and straight-chained, i.e., a straight-chained (linear) olefinic or alkenyl group. The unsaturation occurs by the presence of one or more carbon-carbon double bonds and/or one or more carbon-carbon triple bonds. Some examples of straight-chained olefinic groups include vinyl, propen-1-yl (allyl), 3-buten-1-yl (CH₂═CH—CH₂—CH₂—), 2-buten-1-yl (CH₂—CH═CH—CH₂—), butadienyl, 4-penten-1-yl, 3-penten-1-yl, 2-penten-1-yl, 2,4-pentadien-1-yl, 5-hexen-1-yl, 4-hexen-1-yl, 3-hexen-1-yl, 3,5-hexadien-1-yl, 1,3,5-hexatrien-1-yl, 6-hepten-1-yl, ethynyl, propargyl (2-propynyl), 3-butynyl, and the numerous other straight-chained alkenyl or alkynyl groups having up to 12 carbon atoms.

In a fifth set of embodiments, the hydrocarbon group (R) is unsaturated and branched, i.c., a branched olefinic or alkenyl group. Some examples of branched olefinic groups include propen-2-yl (CH₂═C.—CH₃), 1-buten-2-yl (CH₂=C.—CH₂—CH₃), 1-buten-3-yl (CH₂═CH—CH.—CH₃), 1-propen-2-methyl-3-yl (CH₂═C(CH₃)—CH₂—), 1-penten-4-yl, 1-penten-3-yl, 1-penten-2-yl, 2-penten-2-yl, 2-penten-3-yl, 2-penten-4-yl, and 1,4-pentadien-3-yl, and the numerous other branched alkenyl groups having up to 12 carbon atoms, wherein the dot in any of the foregoing groups indicates a point of attachment.

In a sixth set of embodiments, the hydrocarbon group (R) is unsaturated and cyclic, i.e., a cycloalkenyl group. The unsaturated cyclic group can be aromatic or aliphatic. Some examples of unsaturated cyclic hydrocarbon groups include cyclopropenyl, cyclobutenyl, cyclopentenyl, cyclopentadienyl, cyclohexenyl, cyclohexadienyl, phenyl, benzyl, cycloheptenyl, cycloheptadienyl, cyclooctenyl, cyclooctadienyl, and cyclooctatetraenyl groups. The unsaturated cyclic hydrocarbon group may or may not also be a polycyclic group (such as a bicyclic or tricyclic polyaromatic group) by either possessing a bond between two of the ring groups (e.g., biphenyl) or a shared (i.e., fused) side, as in naphthalene, anthracene, phenanthrene, phenalene, or indene fused ring systems. All of the foregoing cyclic groups are carbocyclic groups. However, the cyclic groups may contain one or more ring heteroatoms selected from N, O, and S atoms to result in a heteroaromatic ring or ring system, e.g., pyridinyl, imidazolyl, piperazinyl, and pyrrolidinyl groups.

Linking groups (—R—) can be derived from any of the above hydrocarbon groups by removing a hydrogen atom therefrom. For example, a hydrogen atom may be removed from a methyl (—CH₃) group to become a methylene (—CH₂—) linker, and similarly, a hydrogen atom may be removed from a phenyl group to become an o-, m-, or p-phenylene linker.

In one aspect, the invention is directed to specialized oxyanion precipitating compounds having a bis(iminoguanidinium) (i.e., “BIG”) structure. These BIG ligands can effectively remove oxyanions from aqueous solutions through crystallization by taking advantage of the low aqueous solubilities of the corresponding oxyanion salts. These ligands, functionalized with guanidinium hydrogen bonding groups, are complementary to oxyanions, such as sulfate, selenate, selenite, chromate, and carbonate, chelating their O-X-O edges or O vertices via strong bidentate hydrogen bonds.

The oxyanion precipitating compounds are within the scope of the following generic structure:

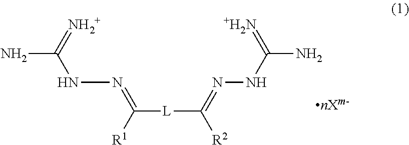

The variable L in Formula (1) is a bond or a hydrocarbon linker containing 1-12 carbon atoms and optionally containing one or more heteroatoms selected from O, N, and S, as described above for the hydrocarbon groups (R). In one set of embodiments, L is a linker. In another set of embodiments, L is a hydrocarbon linker (—R—). In some embodiments, L is composed of only carbon and hydrogen atoms. In other embodiments, L is composed of carbon and hydrogen atoms and is optionally substituted with one or more fluorine atoms. In other embodiments, L contains carbon and hydrogen atoms, and optionally one or more fluorine atoms, along with at least one heteroatom selected from N, O, and S. In particular embodiments, L is a cyclic (ring) linker, which may be carbocyclic or heterocyclic, as described above for R. In other embodiments, L is a linear or branched alkyl or alkenyl group containing 1-12 carbon atoms, or L may be an alkyl-cyclic-alkyl linker, such as a —CH₂—C₆H₄—CH₂— linker.

In some embodiments, L is a ring-containing moiety. The ring-containing moiety is or includes any cyclic group that includes at least one, two, three, or four carbon ring atoms. Since the cyclic group is attached to two iminoguanidinium groups, the cyclic group in the ring-containing moiety necessarily includes two sites engaged in bonds, either directly, or indirectly via a linker, to the iminoguanidinium groups. Typically, the two sites in the ring linked to the iminoguanidinium groups are ring carbon atoms. In some embodiments, the ring-containing moiety is or includes a monocyclic ring, i.e., a single ring not bound or fused to another ring. In other embodiments, the ring-containing moiety is or includes a ring system, wherein the term “ring system” refers to a polycyclic moiety (e.g., a bicyclic or tricyclic moiety). The cyclic group can be polycyclic by either possessing a bond between at least two rings or a shared (i.e., fused) bond between at least two rings. The one or more rings in the ring-containing moiety is typically a five-membered, six-membered, or seven-membered ring.

In the case where L is a ring, Formula (1) includes any regioisomers that may differ in the connection points of the two iminoguanidinium groups on the ring. Thus, as an example, if L is taken as a benzene (phenylene) ring, the two shown iminoguanidinium groups may be located at the 1,4 (para), 1,3 (meta) or 1,2 (ortho) positions. In some embodiments, the iminoguanidinium groups are located the farthest from each other on the ring-containing moiety. In the case of a benzene ring, the farthest positions correspond to the 1,4 (para) positions.

In one set of embodiments, the ring-containing moiety in L is or includes a carbocyclic ring or ring system. The term “carbocyclic” indicates that the ring or ring system contains only ring carbon atoms. The carbocyclic ring or ring system can be saturated or unsaturated. Some examples of carbocyclic rings that are monocyclic and saturated include cyclopentyl, cyclohexyl, and cycloheptyl rings. Some examples of carbocyclic rings that are monocyclic and unsaturated (which may be aliphatic or aromatic) include cyclopentenyl, cyclopentadienyl, cyclohexenyl, cyclohexadienyl, cycloheptenyl, cycloheptadienyl, and phenylene (benzene) rings. Some examples of carbocyclic rings that are polycyclic and saturated include decalin, norbornane, bicyclohexane, and 1,2-dicyclohexylethane ring systems. Some examples of carbocyclic rings that are polycyclic and unsaturated include naphthalene, anthracene, phenanthrene, phenalene, and indene ring systems.

In another set of embodiments, the ring-containing moiety in L is or includes a heterocyclic ring or ring system. The term “heterocyclic” indicates that the ring or ring system contains at least one ring heteroatom. The ring heteroatom is typically selected from nitrogen, oxygen, and sulfur. The heterocyclic ring or ring system can be saturated or unsaturated. Some examples of heterocyclic saturated rings or ring systems include those containing at least one ring nitrogen atom (e.g., pyrrolidine, piperidine, piperazine, imidazolidine, azepane, and decahydroquinoline rings); those containing at least one ring oxygen atom (e.g., oxcetane, tetrahydrofuran, tetrahydropyran, 1,4-dioxane, 1,3-dioxane, and 1,3-dioxepane rings); those containing at least one ring sulfur atom (e.g., tetrahydrothiophene, tetrahydrothiopyran, 1,4-dithiane, 1,3-dithiane, and 1,3-dithiolane rings); those containing at least one ring oxygen atom and at least one ring nitrogen atom (e.g., morpholine and oxazolidine rings); and those containing at least one ring nitrogen atom and at least one ring sulfur atom (e.g., thiazolidine and thiamorpholine rings). Some examples of heterocyclic unsaturated rings or ring systems include those containing at least one ring nitrogen atom (e.g., pyrrole, imidazole, pyrazole, pyridine, pyrazine, pyrimidine, 1,3,5-triazine, azepine, diazepine, indole, purine, benzimidazole, indazole, 2,2′-bipyridine, quinoline, isoquinoline, phenanthroline, 1,4,5,6-tetrahydropyrimidine, 1,2,3,6-tetrahydropyridine, 1,2,3,4-tetrahydroquinoline, quinoxaline, quinazoline, pyridazine, cinnoline, and 1,8-naphthyridine rings); those containing at least one ring oxygen atom (e.g., furan, pyran, 1,4-dioxin, benzofuran, dibenzofuran, and dibenzodioxin); those containing at least one ring sulfur atom (e.g., thiophene, thianaphthene, benzothiophene, thiochroman, and thiochromene rings); those containing at least one ring oxygen atom and at least one ring nitrogen atom (e.g., oxazole, isoxazole, benzoxazole, benzisoxazole, oxazoline, 1,2,5-oxadiazole (furazan), and 1,3,4-oxadiazole rings); and those containing at least one ring nitrogen atom and at least one ring sulfur atom (e.g., thiazole, isothiazole, benzothiazole, benzoisothiazole, thiazoline, and 1,3,4-thiadiazole rings).

The variables R¹ and R² in Formula (1) are independently selected from H and hydrocarbon groups (R) containing 1-12 carbon atoms, as described earlier above. In some embodiments, R¹ and/or R² is/are composed of only carbon and hydrogen atoms. In other embodiments, R¹ and/or R² is/are selected from partially or fully fluorinated hydrocarbon groups. In other embodiments, R¹ and/or R² contains carbon and hydrogen atoms, and optionally one or more fluorine atoms, along with at least one heteroatom selected from N, O. and S. In different embodiments, R¹ and R² independently contain 1, 2, 3, 4, 5, 6, 7, 8, 9, 10, 11, or 12 carbon atoms, or a number of carbon atoms within a range bounded by any two of the foregoing numbers, e.g., 1-10, 1-8, 1-6, 1-4, 1-3, 2-12, 2-10, 2-8, 2-6, 2-4, 3-12, 3-10, 3-8, 3-6, 4-12, 4-10, or 4-8 carbon atoms. In some embodiments, R¹ and R² are selected from linear or branched alkyl or alkenyl groups containing 1-12 carbon atoms, such as any of those described above for R. In more particular embodiments. R¹ and R² are selected from H and methyl groups (e.g., both of R¹ and R² may be H; or one is H and the other methyl; or both R¹ and R² are methyl).

X^{m-} is an anionic species with a magnitude of charge m, where m is an integer of at least 1, and n is an integer of at least 1, provided that n×m=2. For purposes of the invention, X^{m-} is an anionic species exchangeable with sulfate and the toxic oxyanion in the aqueous source before the oxyanion precipitating compound contacts the aqueous source in step (i), and X^{m-} represents a combination of sulfate (e.g., SO₄²⁻) and the toxic oxyanion after the oxyanion precipitating compound contacts the aqueous source. The anionic species before contacting the aqueous source may be any anionic species that can be exchanged for sulfate and the toxic oxyanionic species desired to be removed from the aqueous solution.

In some embodiments, before the oxyanion precipitating compound contacts the aqueous source, the exchangeable anionic species (X^{m-}) is, for example, a halide, such as fluoride, chloride, bromide, or iodide. In other embodiments, the exchangeable anionic species is hydroxide (OH⁻) or an alkoxide (e.g., methoxide or ethoxide). The anionic species can alternatively be a halide equivalent (or pseudohalide), such as methanesulfonate (mesylate), trifluoromethanesulfonate (triflate), tosylate, cyanate, thiocyanate, cyanide, or a sulfonamide anion, such as bis(trifluoromethane)sulfonamide (i.e., bistriflimide). The anionic species may alternatively be a borate anion, such as tetrafluoroborate, tetrakis(pentafluorophenyl)borate, or tetrakis[3,5-bis(trifluoromethyl)phenyl]borate. The anionic species may alternatively be hexafluorophosphate (PF₆⁻). The anionic species may alternatively be a carboxylate species, such as formate, acetate, propionate, or glycolate.

The toxic oxyanion may be selected from any of the known toxic oxyanions, such as selenate (e.g., SeO₄²⁻), selenite (e.g., SeO₃²⁻), chromate (e.g., CrO₄²⁻), and arsenate (AsO₄³⁻). The oxyanions provided above or elsewhere in this disclosure may or may not also include related derivatives. For example, unless otherwise stated, the term “sulfate” may also include thiosulfate (S₂O₃²⁻), bisulfate (HSO₄⁻), and sulfite (SO₃²⁻). Similarly, the term “chromate” may also include Cr₂O₇²⁻ (dichromate). The toxic oxyanion may also be selected from among less common species, such as molybdate (e.g., MoO₄²⁻), tungstate (e.g., WO₄²⁻), arsenate (e.g., AsO₄³⁻), vanadate (e.g., VO₄³⁻), tellurate (e.g., TeO₄²⁻ or TeO₆⁶⁻), and stannate (e.g., SnO₄⁴⁻ or SnO₃²⁻). Any one or more of any of the toxic oxyanionic species disclosed in this application may be present in the aqueous source. In some embodiments, the toxic oxyanion species includes at least selenate and/or selenite.

In embodiments where L is a bond, the oxyanion precipitating compound has the following structure:

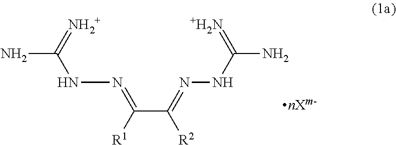

In Formula (1a), R¹ and R² are as defined under Formula (1) including any of the examples provided therein. In some embodiments, one of R¹ and R² is a hydrogen atom while the other is a hydrocarbon group (e.g., methyl), or both of R¹ and R² are hydrogen atoms. In other embodiments, one of R¹ and R² is a hydrocarbon group (e.g., methyl), or both of R¹ and R² are hydrocarbon (e.g., methyl) groups.

In embodiments where L in Formula (1) is or includes a ring, L may be or include an aromatic ring. The aromatic ring may be, for example, a phenylene ring or a heteroaromatic ring containing 1, 2, or 3 nitrogen atoms, such as a pyridinyl ring.

In specific embodiments where L is a phenylene ring, the oxyanion precipitating compound may have the following structure:

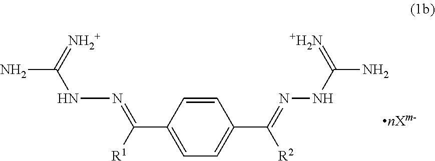

In specific embodiments where L is a pyridinyl ring, the oxyanion precipitating compound may have the following structure:

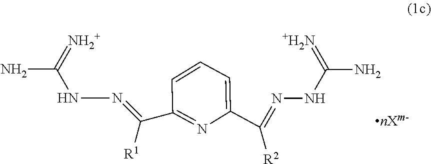

In Formulas (1b) and (1c), R¹ and R² are as defined under Formula (1) including any of the examples provided therein. In some embodiments, one of R¹ and R² is a hydrogen atom while the other is a hydrocarbon group (e.g., methyl), or both of R¹ and R² are hydrogen atoms. In other embodiments, one of R¹ and R² is a hydrocarbon group (e.g., methyl), or both of R¹ and R² are hydrocarbon (e.g., methyl) groups.

Some examples of compounds according to Formulas (1b) and (1c) include the following:

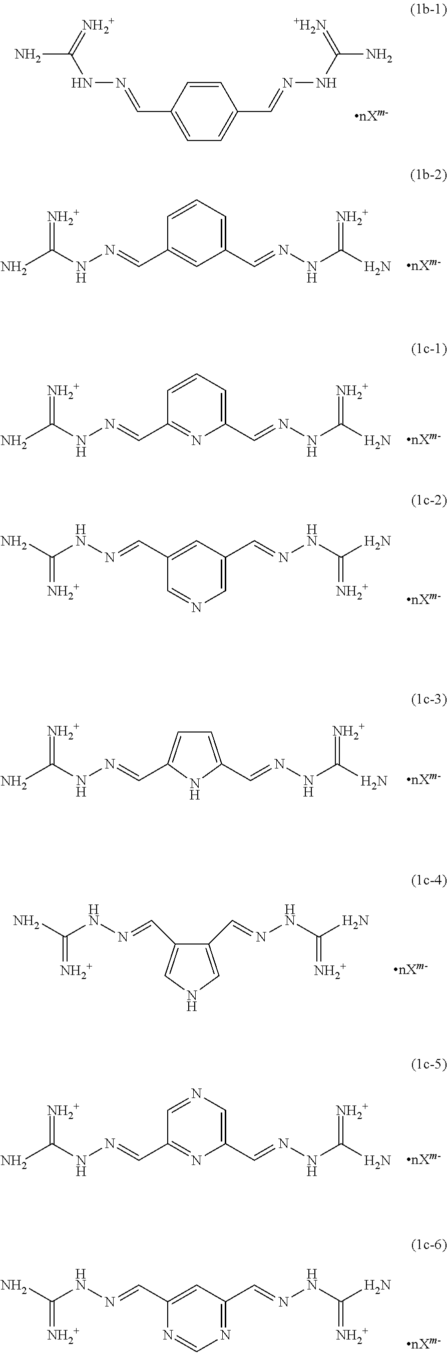

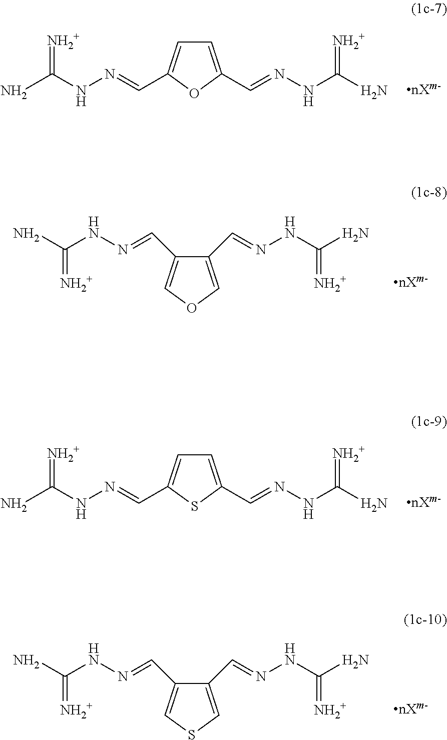 [SMILES: NC(=[NH2+])N/N=C/c1ccc(/C=N/NC(N)=[NH2+])o1 ; NC(=[NH2+])N/N=C/c1cocc1/C=N/NC(N)=[NH2+] ; NC(=[NH2+])N/N=C/c1ccc(/C=N/NC(N)=[NH2+])s1 ; NC(=[NH2+])N/N=C/c1cscc1/C=N/NC(N)=[NH2+]]

Any of the bis(iminoguanidinium) compounds disclosed in this application may be converted to the respective neutral analogue by removal of the two protons located on positively charged amine groups. Moreover, in any of the above exemplary formulas, a hydrogen atom on a ring nitrogen atom may or may not be replaced with a hydrocarbon group, such as a methyl, ethyl, n-propyl, isopropyl, n-butyl, isobutyl, sec-butyl, t-butyl, phenyl, or benzyl group. As also provided above, one or more of the hydrogen atoms in any of the above exemplary structures, whether the hydrogen atoms are shown or not shown, may or may not be replaced with one or more methyl groups.

Although Formula (1) and sub-formulas thereof depict a specific tautomeric arrangement, Formula (1) and sub-formulas can include any other tautomers that can be derived from or interconvert with the tautomer shown in Formula (1). As well known, tautomeric structures have the same atomic connections (aside from one or more protons) but differ in the placement of double bonds, generally with concomitant relocation of one or more protons. Formula (1) and sub-formulas thereof are meant to include all possible tautomers, regioisomers, and stereoisomers. Thus, the positive charge shown in Formula (1) may be located on any of the other nitrogen atoms through tautomerizaton. As well known in the case of tautomers, the positive charge is generally distributed among all atoms capable of holding a positive charge in the various tautomers. Likewise, it is well known that partial double bond character is generally present among all of the bonds capable of engaging in double bonds in the various tautomers. In the event that the structure according to Formula (1) or sub-formula thereof possesses one or more stereocenters, Formula (1) or sub-formula thereof is intended to include all resulting stereoisomers. The stereoisomer may include one or more enantiomers and/or diastereomers.

Some examples of tautomers of Formula (1) are provided as follows:

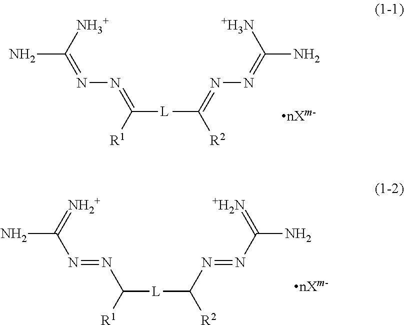

In some embodiments, the oxyanion precipitating compound has any of the following structures:

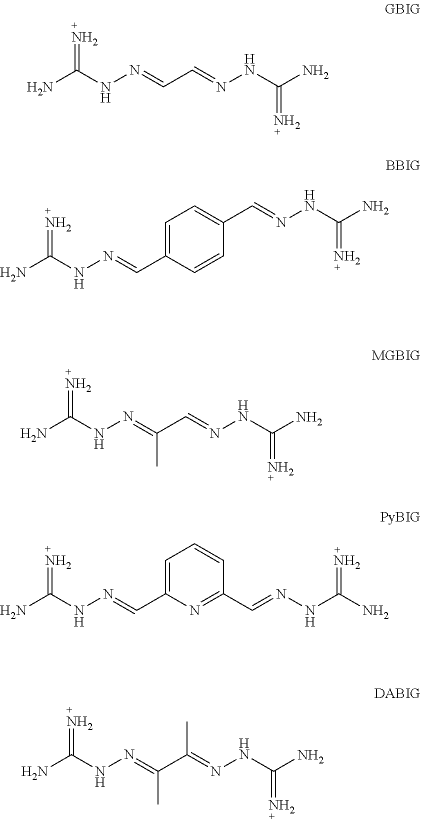 [SMILES: N=C(N)N/N=C/C=N/NC(=N)N ; N=C(N)N/N=C/c1ccc(/C=N/NC(=N)N)cc1 ; CC(/C=N/NC(=N)N)=N\NC(=N)N ; N=C(N)N/N=C/c1cccc(/C=N/NC(=N)N)n1 ; CC(=N\NC(=N)N)/C(C)=N/NC(=N)N]

wherein each of the foregoing structures is associated with an anion of the formula nX^{m-}, as described above.

The compounds according to Formulas (1) sub-formulas thereof can be synthesized by methods well known in the art. In particular embodiments, the compounds according to Formulas (1) and sub-formulas thereof are synthesized by reacting aminoguanidine or aminoguanidinium chloride (or a methylated derivative thereof) with a ring-containing dialdehyde or diketone under conditions where an imine linkage is formed between an amino group on the aminoguanidine or aminoguanidinium molecule and the carbon of the aldehyde or ketone group. The ring-containing dialdehyde or diketone includes a linking portion (A) which may be a bond or hydrocarbon linker, such as a ring-containing moiety, which corresponds to L, as described above. A general schematic of the process is provided as follows:

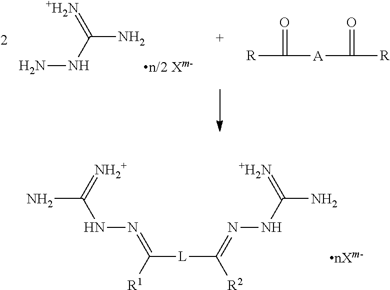

In the above scheme, L is a bond or a hydrocarbon linker (e.g., a ring-containing moiety), as described above. The group R is typically hydrogen (which corresponds to a dialdehyde reactant), but R may independently be a hydrocarbon group, such as methyl, ethyl, n-propyl, isopropyl, n-butyl, isobutyl, sec-butyl, t-butyl, phenyl, or benzyl group, which corresponds to a diketone reactant. Notably, the above scheme is meant to be inclusive of producing a neutral bis-(iminoguanidine) compound that can function as a precursor to the bis-(iminoguanidinium) compounds according to Formula (1). To produce the neutral compound, aminoguanidine can be used in the above scheme instead of a guanidinium salt, or alternatively, the guanidinium salt can be produced and converted to the neutral guanidine compound by reaction with a base. Moreover, any one or more hydrogen atoms of the aminoguanidine or aminoguanidinium reactant may be replaced with one or more methyl groups, respectively, except that the aminoguanidine or aminoguanidinium reactant should retain at least one primary amine group for reaction with the dialdehyde or diketone. Alternatively, one or more hydrogen atoms of the bis-iminoguanidinium or bis-iminoguanidine product may be replaced with one or more methyl groups by, for example, reaction with methyl iodide.

In another aspect, the invention is directed to a method for removing one or more toxic oxyanions from an aqueous source containing sulfate and the one or more toxic oxyanions by use of any of the above-described oxyanion precipitating compounds. The one or more toxic oxyanions may be selected from any of those mentioned in this application, such as, for example, one or more of selenate, selenite, chromate, molybdate, tungstate, arsenate, and stannate. For purposes of the invention, the sulfate in the aqueous source is in a molar concentration equal to or greater than (e.g., 1.5, 2, 3, 4, 5, 10, 20, 50, 100, 200, or 500 times) the total molar concentration of the toxic oxyanion. The aqueous source can be any source containing one or more toxic oxyanions to be removed. The aqueous source may be, for example, drinking water, ground water, municipal wastewater, or industrial wastewater or effluent (e.g., leachate from coal ash). The aqueous source may or may not contain sulfate, or the aqueous source may contain sulfate below a necessary or optimal level. In either case, sulfate can be added to the aqueous source to raise the sulfate concentration to a proper level, as further discussed below. In some embodiments, the solvent portion of the aqueous source is composed completely of water. In other embodiments, the solvent portion of the aqueous source is predominantly composed of water (e.g., at least or above 60, 70, 80, 90, 95, or 99 vol %) and includes an organic solvent miscible in water, such as an alcohol, acetone, acetonitrile, NMP, DMF, DMSO, THE, or the like.

In the method, an oxyanion precipitating compound, such as any of those described in this application within the scope of Formula (1), (1a), (1b), or (1c), is dissolved in the aqueous source containing sulfate and the one or more toxic oxyanions. Upon dissolution of the oxyanion precipitating compound in the aqueous source, the oxyanion precipitating compound forms an insoluble salt with the sulfate and toxic oxyanion(s), wherein the sulfate and toxic oxyanion(s) are co-crystallized. The oxyanion precipitating compound is included in the aqueous source in a molar concentration equal to or greater than (e.g., 2, 3, 4, or 5 times) the total molar concentration of sulfate and toxic oxyanion(s).

The anionic species (X^{m-}) in Formula (1) or sub-formula thereof, before contact with the aqueous source, is exchangeable with the oxyanion to be removed from the aqueous source. In some embodiments, the anionic species in Formula (1) or sub-formula thereof, before contact with the aqueous source, is more specifically a halide, pseudohalide, hydroxide, or an alkoxide. After the oxyanion precipitating compound is dissolved in the aqueous source, the anionic species (X^{m-}) in Formula (1) or sub-formula thereof represents sulfate oxyanion co-crystallized with and any one or more of the toxic oxyanions described above.

As noted above, in the method for removing one or more toxic oxyanions from an aqueous source, the oxyanion precipitating compound is first dissolved in the aqueous source. The foregoing dissolution process can be referred to as “step (i).” The oxyanion precipitating compound refers to any of the bis(iminoguanidinium) compounds of Formula (1) or sub-formula thereof wherein the anionic species (X^{m-}) is exchangeable with the oxyanion to be removed from the aqueous source. That is, the anionic species (X^{m-}) in the bis-iminoguanidinium compound of Formula (1) or sub-formula thereof, before being contacted with and dissolved in the aqueous source, should be capable of being replaced with the sulfate and toxic oxyanions to be removed from the aqueous source. The oxyanion precipitating compound can be dissolved in the aqueous source by any suitable means, such as by directly adding solid oxyanion precipitating compound to the aqueous source or by adding a pre-made solution, suspension, or slurry of the oxyanion precipitating compound to the aqueous source.

The oxyanion precipitating compound is added to the aqueous source in such amount and under such conditions (e.g., temperature) that result in precipitation of a salt containing sulfate, the toxic oxyanion(s), and the oxyanion precipitating compound. Typically, the process is conducted under room temperature (typically 20-30° C., or about 25° C.) and standard pressure (approximately 1 atm). The term “precipitation,” as used herein, refers to the separation of the salt, as a solid, from the aqueous source. The precipitate can be, for example, an amorphous solid (e.g., as scale, sludge, or powder) or crystalline material. In preferred embodiments, the precipitate is in crystalline form since crystal formation functions as an additional driving force for removal of the toxic oxyanion from solution.

Generally, the oxyanion precipitating compound is added to the aqueous source in an amount corresponding to at least, and generally above, the molar amount of total oxyanion (sulfate and toxic oxyanion and any other oxyanion that may be present) expected to be contained within a sample of aqueous source to be processed. As noted earlier above, the oxyanion precipitating compound is typically included in the aqueous source in a molar concentration equal to or greater than (e.g., 2, 3, 4, or 5 times) the total molar concentration of sulfate and toxic oxyanion(s).

In some embodiments, additional sulfate is added to the aqueous source before or simultaneously with dissolution of the oxyanion precipitating compound in the aqueous source, particularly in a situation where the original sulfate content in the aqueous source is below optimal (e.g., below, 50, 100, 200, 300, 400, 500, or 1000 times the total concentration of toxic oxyanion(s)). In a first set of embodiments, the aqueous source initially contains a concentration of sulfate that is less than 100 times the total concentration of toxic oxyanion, and sulfate is added to the aqueous source to result in the concentration of sulfate being at least or more than 100 times the total concentration of toxic oxyanion. In a second set of embodiments, the aqueous source initially contains a concentration of sulfate that is less than 200 times the total concentration of toxic oxyanion, and sulfate is added to the aqueous source to result in the concentration of sulfate being at least or more than 200 times the total concentration of toxic oxyanion. In a third set of embodiments, the aqueous source initially contains a concentration of sulfate that is less than 500 times the total concentration of toxic oxyanion, and sulfate is added to the aqueous source to result in the concentration of sulfate being at least or more than 500 times the total concentration of toxic oxyanion. In a fourth set of embodiments, the aqueous source initially contains a concentration of sulfate that is less than 1000 times the total concentration of toxic oxyanion, and sulfate is added to the aqueous source to result in the concentration of sulfate being at least or more than 1000 times the total concentration of toxic oxyanion.

Following the dissolution of the oxyanion precipitating compound and precipitation of the salt in step (i), the precipitated salt is removed from the aqueous source to result in a supernatant substantially reduced in the concentration of the toxic oxyanion originally present in the aqueous source. The removal step can be referred to as step (ii). The precipitated salt can be removed by any of the means well known in the art for removing solid material from a liquid. The precipitated salt can be removed by, for example, filtration, or by centrifugation followed by decanting, or by a combination thereof. By use of the oxyanion precipitating compounds described herein, the toxic oxyanion salt being removed can be reduced by at least or above 90% compared to the original concentration of the oxyanion in the aqueous source. In embodiments, the supernatant contains at least 90%, 92%, 95%, 96%, 97%, 98%, or 99% lower concentration of the toxic oxyanion compared to the aqueous source.

In first set of embodiments, the aqueous source contains a concentration of toxic oxyanion of at least or up to 1000 ppb and the supernatant in step (ii) contains a toxic oxyanion concentration of no more than or less than 20 ppb, 50 ppb, or 100 ppb. In a second set of embodiments, the aqueous source contains a concentration of toxic oxyanion of at least or up to 500 ppb and the supernatant in step (ii) contains a toxic oxyanion concentration of no more than or less than 2 ppb, 5 ppb, 10 ppb, 20 ppb, or 50 ppb. In a third set of embodiments, the aqueous source contains a concentration of toxic oxyanion of at least or up to 200 ppb and the supernatant in step (ii) contains a toxic oxyanion concentration of no more than or less than 1 ppb, 2 ppb, 5 ppb, 10 ppb, or 20 ppb. In a fourth set of embodiments, the aqueous source contains a concentration of toxic oxyanion of at least or up to 100 ppb and the supernatant in step (ii) contains a toxic oxyanion concentration of no more than or less than 1 ppb, 2 ppb, 5 ppb, or 10 ppb. In a fifth set of embodiments, the aqueous source contains a concentration of toxic oxyanion of at least or up to 50 ppb and the supernatant in step (ii) contains a toxic oxyanion concentration of no more than or less than 1 ppb, 2 ppb, or 5 ppb.

In some embodiments, the method further includes a recycling step of the oxyanion precipitating compound as follows: (iii) treating the salt obtained in step (ii) with a base (e.g., a metal hydroxide, organic amine, or ammonia) to form an uncharged (neutral) precursor of the oxyanion precipitating compound along with simultaneous release and isolation of sulfate and toxic oxyanions. The neutral precursor of the oxyanion precipitating compound can have the following structure:

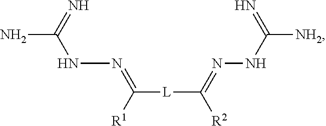

wherein R¹ and R² are as defined earlier above. The neutral form may also have any of the different tautomeric forms discussed above for the oxyanion precipitating compounds of Formula (1) and sub-formulas thereof.

The recycling by reaction with a base forms a byproduct salt (e.g., metal sulfate, organoammonium sulfate, or ammonium sulfate, respectively) with the oxyanion originally bound with the oxyanion precipitating compound. To reform the oxyanion precipitating compound according to Formula (1), the uncharged precursor is then acidified with a protic acid of the formula HX (e.g., HCl, HBr, HI, or HNO₃, etc.), with X^{m-} being the conjugate base of the acid used (e.g., Cl⁻, Br⁻, I⁻, or NO₃⁻, respectively).

In separate or further embodiments, to recover and recycle additional oxyanion precipitating compound that may not have been precipitated in the initial contact with the aqueous source (which can occur particularly when excess oxyanion precipitating compound is used), sulfate is added to the supernatant in step (ii) to precipitate additional salt containing the oxyanion precipitating compound. The additional salt is then combined with the salt already removed in step (ii) to form a total amount of precipitated salt. The total combined salt can then be treated by a recycling process as described above, or more particularly as follows: (iii) treating the total amount of precipitated salt with a base to form an uncharged precursor of the oxyanion precipitating compound along with simultaneous release and isolation of sulfate and toxic oxyanion, followed by acidification of the uncharged precursor of the oxyanion precipitating compound with an acid of the formula HX, wherein X is an anionic species exchangeable with sulfate and the toxic oxyanion in the aqueous source to re-form the oxyanion precipitating compound of Formula (1)

Examples have been set forth below for the purpose of illustration and to describe certain specific embodiments of the invention. However, the scope of this invention is not to be in any way limited by the examples set forth herein.

### EXAMPLES

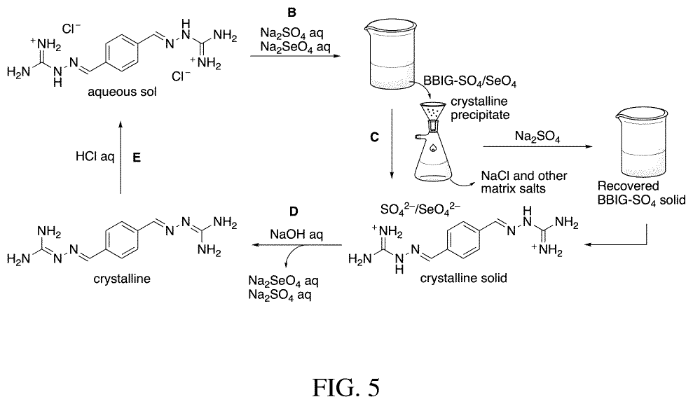

In a typical experiment, BIG chloride was added to wastewater containing sulfate, selenate, and/or selenite anions, and immediately upon addition, a white precipitate formed. The resulting suspensions were mixed for 24 h using a rotator set at 60 rpm inside an incubator set to 25 C. The samples were then removed and centrifuged for 10 min at 3000 rpm to separate the aqueous and solid phases. In the field, the suspended solids were filtered through a filter press. A 1 mL aliquot was then removed from each sample and passed through a 0.02 μm syringe filter to ensure any suspended solid was removed from the filtrate prior to diluting the samples in 2% HNO₃ for ICP-MS analysis. To recover the BIG ligand, 2.1 equiv. of NaOH (or adjusted to a pH of 11) was added to a suspension of the BIG-oxyanion salts in a 15 mL polypropylene centrifuge tube. The suspension was mixed for 24 h at 60 rpm. The suspended BIG neutral solid was filtered from the supernatant solutions containing the oxyanions dissolved as soluble sodium salts. The BIG chloride salts were then regenerated by the addition of 0.1 M HCl to the neutral ligands. This process is depicted schematically in FIG. 5.

To the initial filtrate containing unreacted BIG, 1.1 equiv. of Na₂SO₄ was added to the solution to recover any remaining BIG. The solutions were mixed for 24 h at 60 rpm, where a white solid formed. The suspended solids were then centrifuged for 10 min at 3000 rpm to separate the aqueous and solid phases. This recovered solid can be added to the Se-containing crystalline solid for treatment with base and acid regeneration to recycle the BIG ligand.

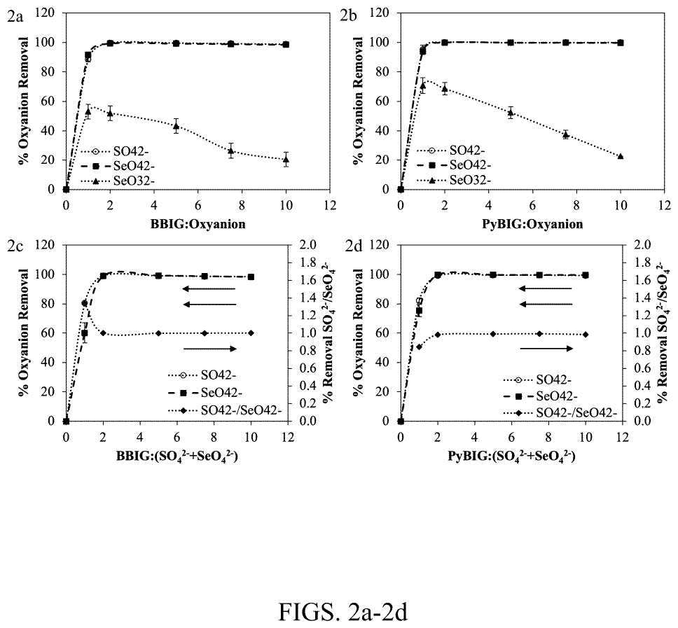

Crystallization studies with BBIG and individual sulfate, selenate, or selenite anions (5 mM) were performed, while the concentration of BIGs was increased from one to ten molar equivalents. Starting with BBIG, as shown in FIG. 2a, the removal of both sulfate and selenate was nearly quantitative after increasing BBIG concentration to twice that of sulfate or selenate, with final sulfate and selenate removal of 99.7 and 99.2%, respectively. On the other hand, only approximately half (53.0%, FIG. 2a) of the selenite was removed from the solution with 1 equivalent of BBIG, which can be attributed to its propensity to form HScO₃⁻, by deprotonating BBIG in the process. As shown in FIG. 2b, PyBIG removed sulfate and selenate with 95.0% and 93.9% efficiency, respectively, at 5 mM (1 equiv), and near quantitatively (99.9% and 99.8%, respectively) at 10 mM. Once again, selenite was removed to a lesser extent (70.7%) with 1 equivalent of PyBIG (FIG. 2b).

Concomitant crystallization of sulfate and selenate with either BBIG or PyBIG led to near quantitative removal of both oxyanions from equimolar aqueous mixtures. Cocrystallization experiments were carried out keeping the concentration of sulfate and selenate at near equimolar concentrations of 2.5 mM, while the BIG concentrations were increased gradually from 5 to 50 mM. As shown in FIG. 2c, with 1 molar equivalent of BBIG relative to the total oxyanion concentration, 80.6 and 60.2% of sulfate and selenate are removed, respectively. Increasing the BBIG concentration to 2 equivalents resulted in the highest removal efficiencies of 99.2% and 99.0% for sulfate and selenate, respectively. At higher BBIG concentrations, the oxyanion removal remained near quantitative. Referring to the ratio of sulfate to selenate % removal, a ratio of 1.0 indicates there is no preference for sulfate over selenate. At 1 equivalent of BBIG, the removal of 1.34 indicates that sulfate is slightly preferred. At relative ligand concentrations higher than 1 equivalent of BBIG, the ratio of removal efficiencies approaches 1, indicating no preference between the two oxyanions by crystallization under these conditions.

PyDIG also quantitatively removed sulfate and selenate at higher BIG concentrations. As shown in FIG. 2d, the SO₄²⁻ and SeO₄²⁻ anions are only 82.3% and 75.6% removed, respectively, with 1 equivalent of PyBIG. Increasing the PyBIG concentration resulted in 97.8 and 99.7% sulfate and selenate removal, respectively, which plateaus after 2 equivalents of PyBIG. The sulfate-to-selenate removal efficiency is 0.84 at 1 equivalent of PyBIG, which indicates that selenate is preferred at this concentration. Increasing the PyBIG concentration achieved a ratio of approximately 1, indicating no discrimination between sulfate and selenate with excess PyBIG. With both BBIG and PyBIG, it appears the 2:1 BIG:oxyanions ratio is ideal for effectively removing SO₄²⁻ and SeO₄²⁻ anions from solution with minimal excess of the BIG ligands.

Recovery of the BIGs from the crystalline solids was carried out through pH swings, by adding a slight excess of NaOH to neutralize the guanidinium cations and converting the insoluble BIG-oxyanion salt to either the BBIG or PyBIG free base, while releasing the bound oxyanion in solution as the sodium salt (FIG. 5). For BBIG, 89.6% of 90.3% of the cocrystallizing sulfate and selenate were released in solution, respectively, when present as single oxyanion salts. The release rates from the crystalline solid do not change when both anions are present in the solid. Relative to the initial BBIG-oxyanion precipitate, 84.1±1.9% BBIG free base was recovered. The releases of sulfate and selenate are slightly higher with PyBIG, with measured values greater than 95%. As a result of its higher solubility, the recovery of PyBIG free base is 23.2±4.0%. After isolation of the neutral big species, treatment with dilute acid produces the BIG chloride salts, thereby completing the separation cycles for sulfate and selenate removal.

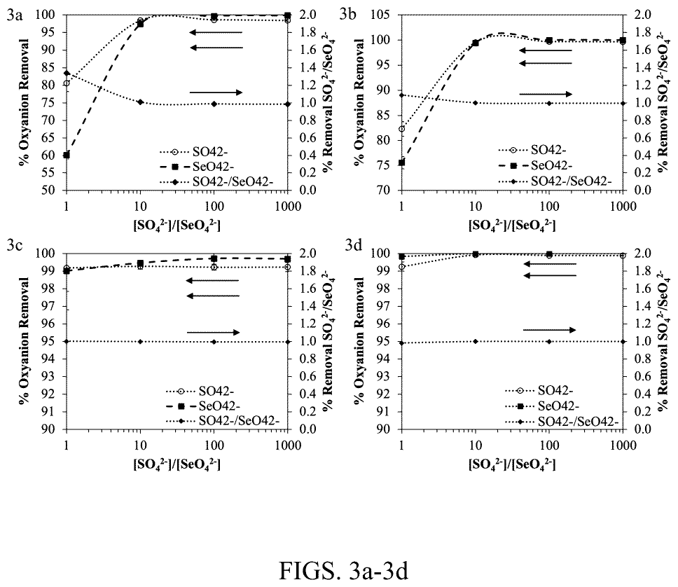

Crystallization experiments were carried out keeping sulfate at a constant concentration and decreasing the selenate concentration while maintaining constant ionic strength to mimic wastewater conditions. These experiments were carried out at 1 and 2 molar equivalents of BBIG or PyBIG. The sulfate concentration was kept constant at 2.5 mM, while the selenate concentration was incrementally reduced from 2.5 mM to 2.5 μM. As indicated in FIG. 3a, with 1 equivalent of BBIG, and 2.5 mM each of sulfate and selenate, an initial preference for sulfate over selenate was observed as discussed earlier. At lower selenate concentrations of 250, 25, and 2.5 μM, there is no discernible preference between the oxyanions. As shown in FIG. 3c, in the presence of 2 equiv of BBIG, oxyanion removal was near quantitative, at 99.2% and 99.4% removal for sulfate and selenate, respectively, with no preference for either oxyanion. At the lowest concentration of selenate, the amount remaining in solution after crystallization is below the limit of detection of 0.02 ppb Se. The same trend is observed for PyBIG, with near quantitative removal of both sulfate and selenate. As shown in FIG. 3b, with 1 equiv of PyBIG, sulfate and selenate were removed at 82.3% and 75.6%, with a slight preference for sulfate over selenate. As shown in FIG. 3d, increasing the amount of PyBIG to 2 equiv results in >99.7% removal of both oxyanions with no preference for sulfate over selenate.

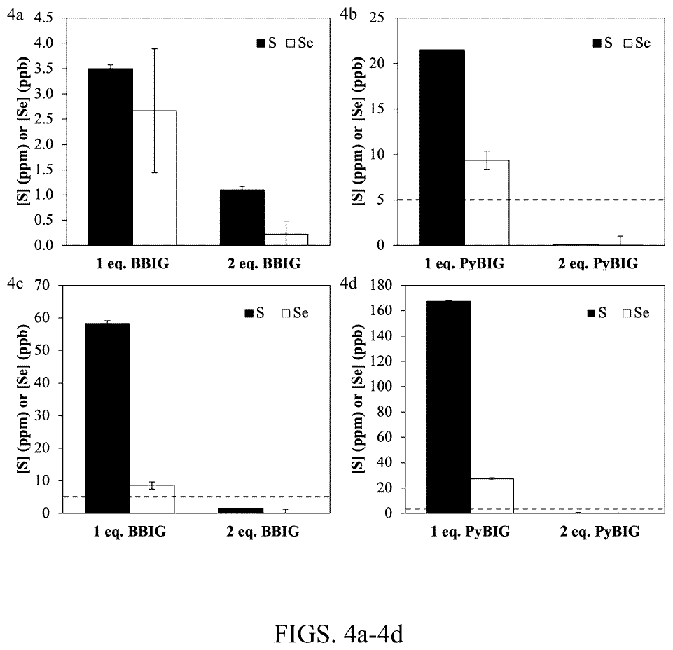

The efficacies of sulfate and selenate removal with BBIG and PyBIG ligands were next tested with simulated wastewater to determine if the EPA's 5 ppb discharge limit was achievable. The initial concentrations of sulfate and selenate in the simulated wastewater were based on typical values found in groundwaters monitored at the Bull Run steam plant of Tennessee Valley Authority (TVA). The simulated Bull Run groundwater contained an 8985-fold excess concentration of sulfate relative to selenate, with sulfate and selenate concentrations of 5.84±0.11 mM and 0.65±0.07 μM, respectively. Cocrystallization studies were performed with 1 and 2 mol equiv of BBIG or PyBIG. As shown in FIG. 4a, with 1 equiv of BBIG added, 98.1% and 94.8% of sulfate and selenate were removed, respectively, resulting in a final Se concentration of 2.67±1.22 ppb. On the other hand, selenate removal was near quantitative with 2 equiv of BBIG (FIG. 4a). Specifically, as shown in FIG. 4c. 99.4% sulfate and 99.6% selenate were removed from solution, with the concentration of selenium reduced to 0.22±0.26 ppb. As shown in FIG. 4b, in the case of PyBIG, 1 equiv can remove 88.5% and 81.6% sulfate and selenate, respectively, to a final Se concentration of 9.39±0.34 ppb. As shown in FIG. 4d, 2 equiv of PyBIG removed 99.94% and 99.96% sulfate and selenate, respectively, down to the limit of detection for Se of 0.02±0.01 ppb.

A more concentrated simulated wastewater solution based on Bull Run's leachate water that has leached from fly ash beds was also tested with BBIG and PyBIG. The concentrations of sulfate and selenate in the simulated leachate were 48.49±0.7 mM and 2.37±0.16 μM, respectively, with sulfate concentration being 20,460 times higher than that of selenate. In the case of BBIG, 1 equiv removed 96.3% and 95.4% sulfate and selenate, respectively, while 2 equiv removed sulfate and selenate near quantitatively at 99.90% and 99.94%, respectively, with a final Se concentration of 8.59±0.62 ppb (1 equiv) and 0.20±0.01 ppb (2 equiv). In the case of PyBIG, 89.2% and 85.5% sulfate and selenate, respectively, were removed with 1 equiv, and 99.98% and 99.99%, respectively with 2 equiv. The final Se concentrations after cocrystallization with PyBIG were 27.23±0.92 ppb (1 equiv) and 0.02±0.01 ppb (2 equiv), reaching the limit of detection. In the above experiments with both the groundwater and leachate, the sulfate-to-selenate removal ratios are approximately one, with no oxyanion preference even at such large differences in concentrations between the two oxyanions. In fact, the large excess of sulfate present in the wastewaters appears to help remove trace amounts of selenate by cocrystallization with BBIG or PyBIG, down to sub-ppb levels of Se.

While there have been shown and described what are at present considered the preferred embodiments of the invention, those skilled in the art may make various changes and modifications which remain within the scope of the invention defined by the appended claims.

What is claimed is:

1. A method for removing a toxic oxyanion from an aqueous source containing sulfate and said toxic oxyanion, the method comprising:
(i) dissolving an oxyanion precipitating compound into said aqueous source to result in precipitation of a salt containing sulfate, toxic oxyanion, and oxyanion precipitating compound, wherein the toxic oxyanion is selected from the group consisting of selenate, selenite, molybdate, arsenate, and chromate, and wherein the sulfate in the aqueous source is in a molar concentration equal to or greater than the total molar concentration of the toxic oxyanion, and the oxyanion precipitating compound is included in the aqueous source in a molar concentration equal to or greater than the total molar concentration of sulfate and toxic oxyanion; and
(ii) removing the precipitated salt from the aqueous source to result in a supernatant containing at least 90% lower concentration of the toxic oxyanion compared to the aqueous source;
wherein the oxyanion precipitating compound has the following structure: 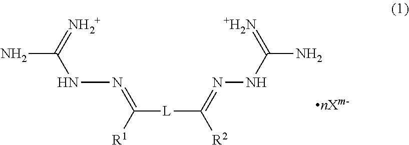
wherein:
L is a bond or a hydrocarbon linker containing 1-12 carbon atoms and optionally containing one or more heteroatoms selected from O, N, and S;
R¹ and R² are independently selected from H and hydrocarbon groups containing 1-12 carbon atoms;
X^{m-} is an anionic species with a magnitude of charge m, where m is an integer of at least 1, provided that X^{m-} is an anionic species exchangeable with sulfate and the toxic oxyanion in said aqueous source before said oxyanion precipitating compound contacts the aqueous source in step (i), and X^{m-} represents a combination of sulfate and the toxic oxyanion after the oxyanion precipitating compound contacts the aqueous source; and
n is an integer of at least 1;
provided that n×m=2.

2. The method of claim 1, wherein L is a bond and the oxyanion precipitating compound has the following structure:

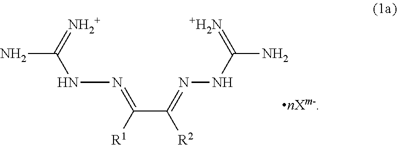

3. The method of claim 2, wherein at least one of R¹ and R² is a methyl group.

4. The method of claim 1, wherein L is an aromatic ring.

5. The method of claim 4, wherein the oxyanion precipitating compound has the following structure:

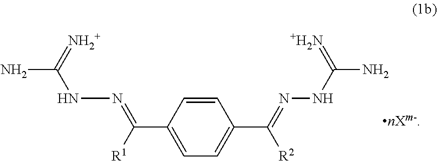

6. The method of claim 4. wherein the oxyanion precipitating compound has the following structure:

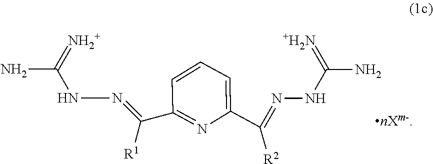

7. The method of claim 1. wherein the oxyanion precipitating compound has any of the following structures: 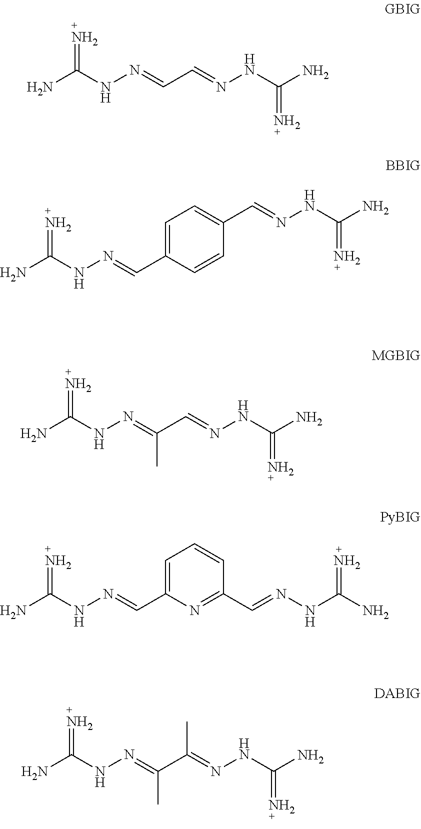 [SMILES: N=C(N)N/N=C/C=N/NC(=N)N ; N=C(N)N/N=C/c1ccc(/C=N/NC(=N)N)cc1 ; CC(/C=N/NC(=N)N)=N\NC(=N)N ; N=C(N)N/N=C/c1cccc(/C=N/NC(=N)N)n1 ; CC(=N\NC(=N)N)/C(C)=N/NC(=N)N]
wherein each of the foregoing structures is associated with an anion of the formula nX^{m-}, as defined in claim 1.

8. The method of claim 1, wherein X^{m-} is a halide species selected from Cl⁻, Br⁻, and I⁻ before said oxyanion precipitating compound contacts the aqueous source in step (i).

9. The method of claim 1, wherein the toxic oxyanion comprises selenate, selenite, or combination thereof.

10. The method of claim 1, further comprising a recycling step of the oxyanion precipitating compound as follows: (iii) treating the salt obtained in step (ii) with a base to form an uncharged precursor of the oxyanion precipitating compound along with simultaneous release and isolation of sulfate and toxic oxyanion, followed by acidification of the uncharged precursor of the oxyanion precipitating compound with an acid of the formula HX, wherein X is an anionic species exchangeable with sulfate and the toxic oxyanion in said aqueous source to re-form the oxyanion precipitating compound of Formula (1).

11. The method of claim 10, wherein X is Cl, Br, or I.

12. The method of claim 1, wherein the sulfate is present in the aqueous source in a concentration at least 100 times the total concentration of toxic oxyanion.

13. The method of claim 1, wherein sulfate is added to the supernatant in step (ii) to precipitate additional salt containing the oxyanion precipitating compound; combining the additional salt with the salt already removed in step (ii) to form a total amount of precipitated salt; and recycling the oxyanion precipitating compound as follows: (iii) treating the total amount of precipitated salt with a base to form an uncharged precursor of the oxyanion precipitating compound along with simultaneous release and isolation of sulfate and toxic oxyanion, followed by acidification of the uncharged precursor of the oxyanion precipitating compound with an acid of the formula HX, wherein X is an anionic species exchangeable with sulfate and the toxic oxyanion in said aqueous source to re-form the oxyanion precipitating compound of Formula (1).

14. The method of claim 1, wherein the oxyanion precipitating compound is included in the aqueous source in at least twice the molar concentration of the total molar concentration of sulfate and toxic oxyanion.

15. The method of claim 1, wherein the aqueous source initially contains a concentration of sulfate that is less than 100 times the total concentration of toxic oxyanion, and sulfate is added to the aqueous source to result in the concentration of sulfate being at least 100 times the total concentration of toxic oxyanion.

16. The method of claim 1, wherein the aqueous source initially contains a concentration of sulfate that is less than 500 times the total concentration of toxic oxyanion, and sulfate is added to the aqueous source to result in the concentration of sulfate being at least 500 times the total concentration of toxic oxyanion.

17. The method of claim 1, wherein the aqueous source is drinking water.

18. The method of claim 1, wherein the aqueous source contains a concentration of toxic oxyanion of at least 500 ppb and the supernatant in step (ii) contains a toxic oxyanion concentration of no more than 50 ppb.

19. The method of claim 1, wherein the aqueous source contains a concentration of toxic oxyanion of at least 200 ppb and the supernatant in step (ii) contains a toxic oxyanion concentration of no more than 20 ppb.

20. The method of claim 1, wherein the aqueous source contains a concentration of toxic oxyanion of at least 100 ppb and the supernatant in step (ii) contains a toxic oxyanion concentration of no more than 10 ppb.

21. The method of claim 1, wherein the aqueous source contains a concentration of toxic oxyanion of at least 50 ppb and the supernatant in step (ii) contains a toxic oxyanion concentration of no more than 5 ppb.
